(19) United States
(12) Patent Application Publication
(10) Pub. No.: US 2016/0283880 A1
(43) Pub. Date: Sep. 29, 2016

(54) HEALTH CARE ENTERPRISE RESOURCE PLANNING SYSTEM AND METHOD FOR UTILIZING WEB DATA

(71) Applicant: McKesson Corporation, San Francisco, CA (US)

(72) Inventor: Junyi Jin, Clarendon Hills, IL (US)

(21) Appl. No.: 14/671,248

(22) Filed: Mar. 27, 2015

(51) Int. Cl. G06Q 10/06; G06F 19/00; G06Q 30/02; G06Q 50/22

(52) U.S. Cl. CPC G06Q 10/06313; G06Q 50/22; G06F 19/327; G06Q 30/0202; G06Q 10/06315

(57) ABSTRACT

A health care enterprise resource planning system, method and computer program product are provided in order to more accurately engage in resource planning. In regards to a health care enterprise resource planning system, processing circuitry is provided that is configured to receive web data relating to one or more health-related conditions that are capable of modifying a demand for resources of a health care organization. The processing circuitry is also configured to determine a correlation between the web data and the demand for resources of the health care organization. The processing circuitry is further configured to modify an allotment of resources of the health care organization at least partially based upon the correlation.

### TECHNOLOGICAL FIELD

A health care enterprise resource planning system and method are provided in accordance with an example embodiment of the present invention and, more particularly, a health care enterprise resource planning system, method and computer program product for determining the allotment of resources of a health care organization based at least partially upon web data.

### BACKGROUND

Health care organizations, such as hospitals, rehabilitation facilities and other types of medical facilities, engage in resource planning in order to allot a sufficient quantity of resources at an appropriate time in order to service the patients of the health care organization. In this regard, health care organizations engage in resource planning with respect to physical resources, such as beds and various medical supplies that are utilized to treat patients, but also with respect to human resources, such as the different types of health care practitioners who are needed to properly care for the patients. In this regard, the resource planning activities of a health care organization strive to allocate sufficient resources to care for the anticipated volume of patients, but not to allocate an excessive quantity of resources since resources that go unconsumed in such circumstances create adverse consequences including, for example, a potential reduction in the financial performance of the health care organization, spoilage of some of the supplies, etc.

However, the current resource planning techniques employed by health care organizations are not generally sufficiently accurate. Thus, current resource planning techniques may allocate too few resources or too many resources, particularly as demand deviates from its historical averages. Thus, improvements in the resource planning techniques employed by health care organizations would be desirable.

### BRIEF SUMMARY

A health care enterprise resource planning system, method and computer program product are provided in accordance with an example embodiment in order to more accurately engage in resource planning. In this regard, the health care enterprise resource planning system, method and computer program product at least partially base the resource planning upon web data, such as search results and/or information gleaned from social media sites, that provide information regarding health-related conditions that is more temporally relevant than the historical data of a health care organization. As such, the health care enterprise resource planning system, method and computer program product of an example embodiment may deviate from the resource planning otherwise suggested by the historical data based upon the web data and correlations that have been drawn between the web data and the demand for resources of the health care organization, thereby increasing the likelihood that the health care organization will have appropriate resources on hand to address patient demand since the allotment of resources is at least partially based upon more temporally relevant data.

In an example embodiment, a health care enterprise resource planning system is provided that includes processing circuitry configured to receive web data relating to one or more health-related conditions that are capable of modifying a demand for resources of a health care organization. The processing circuitry is also configured to determine a correlation between the web data relating to the one or more health-related conditions and the demand for resources of the health care organization. The processing circuitry is further configured to modify an allotment of resources of the health care organization at least partially based upon the correlation between the web data relating to the one or more health-related conditions and the demand for resources of the health care organization.

The processing circuitry of an example embodiment is configured to receive web data by filtering the web data based upon a geographical location associated with the web data relative to a service area of the health care organization. As such, the web data upon which the correlation is determined is web data that is associated with a geographical location within the service area of the health care organization. The processing circuitry of an example embodiment is configured to receive web data by receiving search results based upon one or more search queries relating to the one or more health-related conditions. Additionally or alternatively, the processing circuitry of an example embodiment is configured to receive web data by identifying information provided via social media relating to the one or more health-related conditions.

The processing circuitry of an example embodiment is configured to determine the correlation based upon a latency between timing associated with the web data and timing associated with the demand for resources of the health care organization. In this example embodiment, the processing circuitry is also configured to modify the allotment of the resources of the health care organization based at least partially upon the latency between the web data relating to one or more health-related conditions and the demand for resources of the health care organization. The processing circuitry of an example embodiment is configured to determine the correlation based upon the latency by determining a delay between a change in the web data relating to the one or more health-related conditions and the change in the demand for resources of the health care organization based upon the historical web data and historical data regarding the demand for resources of the health care organization.

The processing circuitry of an example embodiment is additionally or alternatively configured to determine the correlation by determining a relationship between a change in a quantity of the web data relating to the one or more health-related conditions and a change in the demand for resources of the health care organization based upon historical web data and historical data regarding the demand for resources of the health care organization. The processing circuitry of an example embodiment is configured to modify the allotment of resources of the health care organization by modifying the allotment of at least one of human resources or physical resources of the health care organization that are utilized to treat the one or more health-related conditions.

In another example embodiment, a method of performing enterprise resource planning for a health care organization based at least partially upon web data is provided. The method includes receiving web data relating to one or more health-related conditions that are capable of modifying a demand for resources of the health care organization. The method also includes determining a correlation between the web data relating to the one or more health-related conditions and the demand for resources of the health care organization. The method further includes modifying an allotment of the resources of the health care organization at least partially based upon the correlation between the web data relating to the one or more health-related conditions and the demand for resources of the health care organization.

The method of an example embodiment also filters the web data based upon a geographical location associated with the web data relative to a service area of the health care organization. As such, the web data upon which the correlation is determined is web data that is associated with a geographical location within the service area of the health care organization. The method of an example embodiment receives web data by receiving search results based upon one or more search queries relating to the more or more health-related conditions. Additionally or alternatively, the method of an example embodiment receives web data by identifying information provided via social media relating to the one or more health-related conditions.

The method of an example embodiment determines the correlation based upon a latency between timing associated with the web data and timing associated with the demand for resources of the health care organization. In this example embodiment, the method also modifies the allotment of the resources of the health care organization based at least partially upon the latency between the web data relating to one or more health-related conditions and the demand for resources of the health care organization. The method of an example embodiment determines the correlation based upon the latency by determining a delay between a change in the web data relating to the one or more health-related conditions and a change in the demand for resources in the health care organization based upon the historical web data and historical data regarding the demand for resources of the health care organization.

The method of an example embodiment additionally or alternatively determines the correlation by determining a relationship between a change in a quantity of the web data relating to the one or more health-related conditions and a change in the demand for resources that the health care organization based upon historical web data and historical data regarding the demand for resources of the health care organization. The method of an example embodiment modifies the resources of a health care organization by modifying at least one of human resources or physical resources of the health care organization that are utilized to treat the one or more health-related conditions.

In a further example embodiment, a computer program product is provided for performing enterprise resource planning for a health care organization based at least partially upon web data. The computer program product includes at least one non-transitory computer-readable storage medium having computer-executable program code portions stored therein with the computer-executable program code portions including program code instructions configured to receive web data relating to one or more health-related conditions that are capable of modifying the demand for resources of a health care organization. The computer-executable program code portions also include program code instructions configured to determine a correlation between the web data relating to the one or more health-related conditions and the demand for resources of the health care organization. The computer-executable program code portions further include program code instructions configured to modify the resources of the health care organization at least partially based upon the correlation between the web data relating to the one or more health-related conditions and the demand for resources of the health care organization.

The program code instructions of an example embodiment that are configured to receive web data include program code instructions configured to filter the web data based upon a geographical location associated with the web data relative to a service area of the health care organization. As such, the web data upon which the correlation is determined is web data that is associated with the geographical location within the service area of the health care organization. The program code instructions configured to receive web data include, in an example embodiment, program code instructions configured to receive search results based upon one or more search queries relating to the one or more health-related conditions. Additionally or alternatively, the program code instructions configured to receive web data include, in an example embodiment, program code instructions configured to identify information provided via social media relating to the one or more health-related conditions.

The program code instructions of an example embodiment that are configured to determine the correlation include program code instructions configured to determine the correlation based upon a latency between timing associated with the web data and timing associated with the demand for resources of the health care organization. In this example embodiment, the that are configured to determine the correlation are also configured to modify the allotment of the resources of the health care organization based at least partially upon the latency between the web data relating to one or more health-related conditions and the demand for resources of the health care organization. The program code instructions of an example embodiment that are configured to determine the correlation based upon the latency include program code instructions configured to determine a delay between a change in the web data relating to the one or more health-related conditions and a change in the demand for resources of the health care organization based upon historical web data and historical data regarding the demand for resources of the health care organization. Additionally or alternatively, the program code instructions of an example embodiment that are configured to determine the correlation based upon the latency include program code instructions configured to determine the relationship between a change in the quantity of the web data relating to the one or more health-related conditions and a change in the demand for resources of the health care organization based upon historical web data and historical data regarding the demand for resources of the health care organization.

In another example embodiment, a method is provided that includes receiving web data relating to one or more health-related conditions that are capable of modifying a demand for resources of a health care organization and determining a change in quantity of the web data related to a respective health-related condition. The method of this example embodiment also includes determining a corresponding change in demand for resources of the health care organization that are required due to treatment of patients having the respective health-related condition based upon a predictive model and the change in the quantity of the web data relating to the respective health-related condition. Further, the method includes modifying the allotment of the resources of the health care organization based on the corresponding change. A corresponding health care enterprise resource planning system and computer program products are also provided.

The health care enterprise resource planning system, method and computer program product of an example embodiment also use the predictive model to determine a latency between the change in the quantity of the web data and the corresponding change in the demand for resources of the health care organization, and modify the allotment of resources of the health care organization at a future time based on determined latency. In this example embodiment, the predictive model may also be used to determine a delay between a change in the web data relating to the one or more health-related conditions and a change in the demand for resources of the health care organization based upon historical web data and historical data regarding the demand for resources of the health care organization. Further, the health care enterprise resource planning system, method and computer program product of an example embodiment filter the web data based upon a geographical location associated with the web data relative to a service area of the health care organization such that the web data that changes in quantity is web data that is associated with a geographical location with the service area of the health care organization.

### BRIEF DESCRIPTION OF THE DRAWINGS

Having thus described certain example embodiments of the present disclosure in general terms, reference will hereinafter be made to the accompanying drawings, which are not necessarily drawn to scale, and wherein:

FIG. 1 is a block diagram of a health care enterprise resource planning system that may be specifically configured in accordance with an example embodiment of the present invention;

FIG. 2 is a flow chart illustrating operations performed, such as by the health care enterprise resource planning system of FIG. 1, in accordance with an example embodiment of the present invention; and

FIG. 3 is a graphical representation of the correlation, including the latency, between web data relating to one or more health-related conditions and the demand for resources of a health care organization that is determined in accordance with an example embodiment of the present invention.

### DETAILED DESCRIPTION

Some embodiments of the present invention will now be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information” and similar terms may be used interchangeably to refer to data capable of being transmitted, received and/or stored in accordance with embodiments of the present invention. Thus, use of any such terms should not be taken to limit the spirit and scope of embodiments of the present invention.

A health care enterprise resource planning system, method and computer program product are provided in accordance with an example embodiment in order to allot resources of a health care organization. The health care organization may be any of a variety of different types of health care organizations that allot resources to the treatment of various health-related conditions. For example, the health care organization may be a hospital, a rehabilitation facility or any of a variety of other health care treatment facilities. The resources allotted include both physical resources, such as beds, medical supplies, etc., and human resources including various types of health care practitioners, such as physicians, nurses, physical therapists, radiologists, etc. configured to care for a patient.

A health care organization may maintain historical data regarding the resources consumed by the health care organization over the course of time. This historical data takes into account seasonal variations that occur on a regular basis, as well as other temporal variations in demand that occur on a regular basis, such as based on a day of week or time of day. For example, the historical data may take into account the demand for greater levels of resources in the emergency room on Friday and Saturday evenings than throughout the remainder of the week. In another example, the historical data may identify the average demand for resources for a particular day of the year going back one or more years. Based upon this historical data, the health care organizations may perform the resource planning, thereby aligning the allotment of resources, both presently and in the future, based upon the historical data including regularly occurring temporal variations reflected by the historical data.

However, the historical data reflecting past usage of resources by a health care organization will not take into account fluctuations in demand that occur irregularly or that are otherwise not reflected by the historical data. As such, the historical data cannot serve as a predictor for such irregular instances of patient demand. For example, in an instance in which there is a measles outbreak, the patient demand for resources of the health care organization to address their measles-related symptoms may be substantially greater than the demand in the past when there were many fewer patients suffering from measles. Thus, reliance by the health care organization upon the historical data in an effort to predict the resources required in response to a measles outbreak may cause the allotted resources to fall materially short of the resources that are actually required to address the patient demand, thereby potentially resulting in patient dissatisfaction and increased health care costs associated with longer patient stays with the health care organization

In order to provide for more accurate resource planning by taking into account data that is temporarily relevant, such as data relating to health care conditions from which potential patients currently suffer, the health care enterprise resource planning system, method and computer program product of an example embodiment receive and analyze web data and, in turn, modify the allotment of resources of the health care organization at least partially based upon the web data. Consequently, the health care enterprise resource planning system, method and computer program product of an example embodiment permit the resources of the health care organization to be allotted in a manner that is not simply based upon historical data, but that is at least partially based upon web data relating to one or more health-related conditions from which potential patients currently suffer such that the allotment of resources more closely mirrors the actual present demand, even in instances in which the demand is irregular relative to that predicted by historical data. As such, the health care enterprise resource planning system, method and computer program product facilitate the allotment of sufficient resources of the health care organization to satisfy patient demand, even in an instance in which the patient demand exceeds the patient demand predicted by historical data, while not allotting an excess amount of resources in an instance in which the expected demand will fall short of that predicted by the historical data of the health care organization.

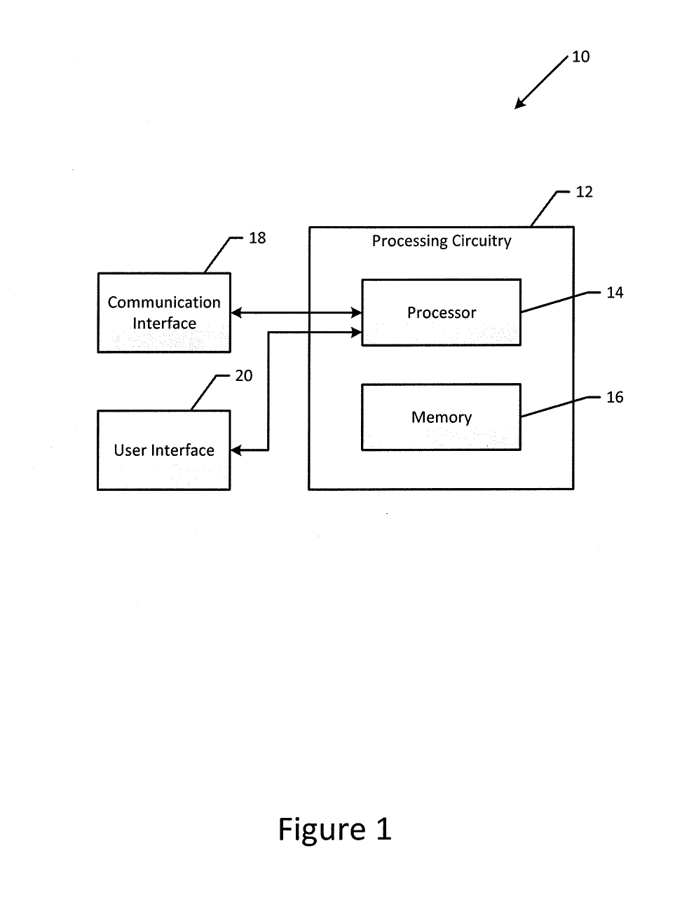

The health care enterprise resource planning system may be embodied in a variety of computer systems, including servers, network computers, personal computers, computer workstations, tablet computers or the like, that are configured to process health care data and to modify the allotment of resources. By way of example, a computer system of an example embodiment that embodies the health care enterprise resource planning system 10 is depicted in FIG. 1 and includes or is otherwise associated and in communication with processing circuitry 12 that is configurable to perform functions in accordance with one or more example embodiments disclosed herein. In this regard, the processing circuitry may be configured to perform and/or control performance of one or more functionalities of the health care enterprise resource planning system or components thereof in accordance with various example embodiments, and thus may provide means for performing functionalities of the computing device. The processing circuitry may be configured to perform data processing, application execution and/or other processing and management services according to one or more example embodiments.

In some example embodiments, the processing circuitry 12 includes a processor 14 and, in some embodiments, such as that illustrated in FIG. 1, further includes memory 16. The processing circuitry may also be in communication with or otherwise control a user interface 18 for communicating with a user and/or a communication interface 20 for communicating with other computing systems. As such, the processing circuitry may be embodied as a circuit chip (e.g., an integrated circuit chip) configured (e.g., with hardware, software or a combination of hardware and software) to perform operations described herein.

The processor 14 may be embodied in a number of different ways. For example, the processor may be embodied as various processing means such as one or more of a central processing unit, a microprocessor or other processing element, a coprocessor, a controller or various other computing or processing devices including integrated circuits such as, for example, an ASIC (application specific integrated circuit), an FPGA (field programmable gate array), or the like. Although illustrated as a single processor, it will be appreciated that the processor may comprise a plurality of processors. The plurality of processors may be in operative communication with each other and may be collectively configured to perform one or more functionalities of the computing device as described herein. The plurality of processors may be embodied on a single computing device or distributed across a plurality of computing devices collectively configured to function as the computing device. In some example embodiments, the processor may be configured to execute instructions stored in the memory 16 or otherwise accessible to the processor. As such, whether configured by hardware or by a combination of hardware and software, the processor may represent an entity (e.g., physically embodied in circuitry—in the form of processing circuitry 12) capable of performing operations according to embodiments of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform one or more operations described herein.

The processing circuitry 12 may also include memory 16 as shown in FIG. 1. In some example embodiments, the memory may include one or more non-transitory memory devices such as, for example, volatile and/or non-volatile memory that may be either fixed or removable. In this regard, the memory may comprise a non-transitory computer-readable storage medium. It will be appreciated that while the memory is illustrated as a single memory, the memory may comprise a plurality of memories. The memory may be configured to store information, data, applications, instructions and/or the like for enabling the computing device to carry out various functions in accordance with one or more example embodiments. For example, the memory may be configured to buffer input data for processing by the processor 14. Additionally or alternatively, the memory may be configured to store instructions for execution by the processor. Among the contents of the memory, applications may be stored for execution by the processor in order to carry out the functionality associated with each respective application. In some cases, the memory may be in communication with the processor via a bus or buses for passing information among components of the health care enterprise resource planning system 10.

In addition to the processing circuitry 12, the health care enterprise resource planning system 10 may include a user interface 18 for displaying and/or receiving data, content or the like. The user interface may include a display, a user input interface or the like. The user input interface, in turn, can include any of a number of devices allowing the computing device to receive data from a user, such as a microphone, a keypad, a touch-sensitive surface (integral or separate from the monitor), a joystick, or other input device. As will be appreciated, the processing circuitry may be directly connected to other components of the computing device, or may be connected via suitable hardware. In one example, the processing circuitry may be connected to the user interface via an adapter configured to permit the processing circuitry to send graphical information to the user interface.

As noted above, the health care enterprise resource planning system 10 of the embodiment of FIG. 1, or components of the health care enterprise resource planning system also include a communication interface 20. Among other functions, the communication interface is configured to communicate with one or more other computer systems, including those accessible via the Internet, in order to collect web data. The communication interface may be any means such as a device or circuitry embodied in either hardware or a combination of hardware and software that is configured to receive and/or transmit messages from sources to subscribers. In this regard, the communication interface may include, for example, an antenna (or multiple antennas) and supporting hardware and/or software for enabling communications with a wireless communication network. Additionally or alternatively, the communication interface may include the circuitry for interacting with the antenna(s) to cause transmission of signals via the antenna(s) or to handle receipt of signals received via the antenna(s). In some environments, the communication interface may alternatively or also support wired communication.

The communication interface 20 may be configured to directly and/or indirectly communicate with other computer systems in any of a number of different manners including, for example, any of a number of wireline or wireless communication or networking techniques. Examples of such techniques include, without limitation, Universal Serial Bus (USB), radio frequency (RF), Bluetooth (BT), infrared (IrDA), any of a number of different cellular (wireless) communication techniques such as any of a number of 2G, 2.5G, 3G, 4G or Long Term Evolution (LTE) communication techniques, local area network (LAN), wireless LAN (WLAN) techniques or the like. In accordance with various ones of these techniques, the communication interface can be coupled to and configured to communicate across one or more networks. The network(s) can comprise any of a number of different combinations of one or more different types of networks, including data and/or voice networks. For example, the network(s) can include one or more data networks, such as a LAN, a metropolitan area network (MAN), and/or a wide area network (WAN) (e.g., Internet), and include one or more voice networks, such as a public-switched telephone network (PSTN).

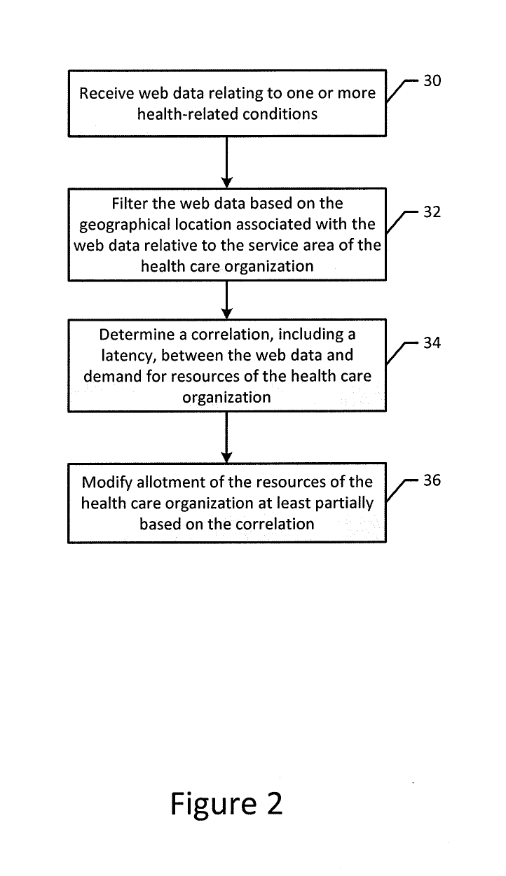

Referring now to FIG. 2, the operations performed, such as by the health care enterprise research planning system 10 of FIG. 1, are depicted. As shown in block 30, the health care enterprise research planning system includes means, such as the processing circuitry 12, the processor 14, the communication interface 20 or the like, for receiving web data relating to one or more health-related conditions that are capable of modifying a demand for resources of a health care organization. The health-related conditions to which the web data relate may be any of a variety of health-related conditions that may be anticipated to cause a potential patient to request the services of the health care organization. As used herein, health-related conditions refer not only to the conditions themselves, but also the symptoms associated with the various conditions, such as fever, dehydration, shortness of breath, etc. regardless of the condition that is causing the symptoms. With respect to a hospital, for example, the health-related conditions may be those that are anticipated to cause a potential patient to be admitted to the hospital or to otherwise seek services from the emergency room or other portion of the hospital. With respect to a rehabilitation facility, the health-related conditions may be those that are anticipated to cause a potential patient to seek rehabilitation services from the health care organization. The health-related conditions to which the web data relates may be irregularly occurring health-related conditions, that is, health-related conditions that occur in an irregular volume or pattern regardless of whether the actual health-related condition is common or unusual. The irregularly occurring health-related conditions therefore occur at an atypical rate that is inconsistent with historical data compiled by the health care organization. For example, the health-related conditions may relate to a measles outbreak experienced by potential patients that deviates materially from historical data.

As described by examples below, the health care enterprise research planning system 10, such as the processing circuitry 12, of an example embodiment actively solicits the web data that is received. For example, the health care enterprise research planning system, such as the processing circuitry, may be configured to specifically request or to otherwise extract the web data from the network, such as the Internet, or a portion thereof. Additionally or alternatively, the health care enterprise research planning system, such as the processing circuitry, may be configured to monitor the network, such as the Internet, or a portion thereof for the web data.

The health care enterprise resource planning system 10, such as the processing circuitry 12, is configured to receive a variety of web data. For example, the health care enterprise resource planning system, such as the processing circuitry, of an example embodiment is configured to receive web data in the form of search results based upon one or more search queries relating to the one or more health-related conditions. The search queries may be predefined (and, as such, may be stored by memory 16) and executed by the processing circuitry, such as in accordance with a predetermined time schedule, in order to search a wide area network, such as the Internet, or a portion thereof for search results that satisfy the one or more search queries. Alternatively, the user may enter a query, such as via the user interface 18, in order to search various forms of social media and to identify information relating to one or more health-related conditions. The search queries may include terminology that relates to a respective health-related condition, including, for example, terminology that relates to symptoms of such a health-related condition, from which a potential patient currently suffers and for which the potential patient is anticipated to seek the services of the health care organization. In another example, the web data that is collected may comprise search terms submitted by potential patients into a search engine, rather than the search results that are returned based on conducting the search.

In a further example, the health care enterprise resource planning system 10, such as the processing circuitry 12, may be configured to acquire web data without concern for the content of the web data, such as by acquiring all web data or a percentage of the entire web data that is communicated via all or a portion of a network. Once acquired, the health care enterprise resource planning system, such as the processing circuitry, of this example embodiment is configured to parse for keywords within the web data. In various embodiments, the parsing may including parsing through all search results or parsing through all search terms or both.

Additionally or alternatively, the health care enterprise resource planning system 10, such as the processing circuitry 12, is configured to receive web data in the form of information provided via social media relating to one or more health-related conditions. Social media is the collective of online communication channels that are dependent upon community-based input to provide user-generated content. For example, social media includes various applications including Facebook, YouTube, Twitter, Instagram, Vine or the like. The health care enterprise resource planning system, such as the processing circuitry, may be configured to query various forms of social media to identify information posted or otherwise provided by users of the social media application that relates to one or more health-related conditions, such as one or more health-related conditions currently experienced by the social media user that are anticipated to cause the social media user to seek the services of the health care organization. As before, the query may be predefined and, as such, may be performed in accordance with a predetermined schedule, such as by the processing circuitry. Alternatively, the user may enter a query, such as via the user interface 18, in order to search various forms of social media and to identify information relating to one or more health-related conditions. As before, the health-related conditions may include irregularly occurring health-related conditions that are not readily predictable from the historical data related to the health care organization.

In an example embodiment, the health care enterprise resource planning system 10 includes means, such as the processing circuitry 12, the processor 14 or the like, for filtering the web data based upon a geographical location associated with the web data relative to a service area of the health care organization. See block 32 of FIG. 2. In this regard, the health care organization is generally configured to serve patients within a service area, such as a portion of a community, e.g., the south side of a city, one or more counties or some other geographical region. In addition, the web data that is received is generally associated with a geographical location. The geographical location associated with web data may be defined in various manners. For example, the web data may contain or be associated with metadata that includes location information relating to the web data. Additionally or alternatively, the geographical location may be based upon the address or other location information associated with a social media user whose information has been received in the form of web data relating to a health-related condition. Alternatively, the geographical location may be based upon the geographical location associated with the computer that is the source of the web data or that serves to store and/or transmit the web data. By filtering the web data based upon the geographical location associated with the web data relative to the service area of the health care organization, the web data that is further analyzed as described below may, in an example embodiment, be only the web data having a geographical location that is within the service area of the health care organization and not web data associated with a geographical location that is outside of the service area of the health care organization. Thus, the web data that is further analyzed as described below relates to potential patients that may seek the services of the health care organization since the potential patients are relatively close to the health care organization, that is, within the service area of the health care organization.

In another embodiment, the health care enterprise resource planning system 10, such as the processing circuitry 12, does not filter out the web data for all geographical regions other than the service area of the health care organization. Instead, the health care enterprise resource planning system, such as the processing circuitry, of this example embodiment also analyzes the web data associated with other regions that are proximate, e.g., adjacent to, the service area of the health care organization. This analysis of the web data associated with proximate regions may be conducted in concert with the analysis of the web data associated with service area of the health care organization or separately from the analysis of the web data associated with service area of the health care organization. By monitoring the web data associated with proximate regions, the health care enterprise resource planning system, such as the processing circuitry, of this example embodiment may adjust the resources, such as by increasing the resources, on hand for the health care organization if the anticipated demand in the proximate regions based upon the web data is unusually high, such as by exceeding the capacity in the proximate regions or by exceeding a predefined percentage of the capacity in the proximate regions, since there may be more overflow than typical from these proximate regions that are anticipated to experience increased demand based upon the web data.

As shown in block 34 of FIG. 2, the health care enterprise resource planning system 10 also includes means, such as the processing circuitry 12, the processor 14 or the like, for determining a correlation between the web data relating to the one or more or health-related conditions and the demand for resources of the health care organization. In other words, the processing circuitry of an example embodiment is configured to determine the impact upon the demand for resources of the health care organization, such as an increase or a decrease in the anticipated demand for resources for which the health care organization has otherwise planned as a result of an analysis of the historical data related to the demand for resources of the health care organization, that is attributable to the web data relating to the one or more health-related conditions. In accordance with an example embodiment, the historical demand relating to the demand for resources of the health care organization may document not only the overall demand for resources, but also the demand for resources that is attributable to each of one or more respective health-related conditions.

By way of example, the processing circuitry 12 of an example embodiment is configured to determine the correlation by determining a relationship between a change in the quantity of the web data relating to the one or more health-related conditions and a change in the demand for resources of the health care organization based upon historical web data and historical data regarding the demand for resources of the health care organization. In this regard, the processing circuitry may determine that a first quantity of web data relating to a respective health-related condition correlated to a first demand for resources as evidenced by the historical data regarding the demand for resources of the health care organization that corresponds in time to the historical web data. By repeating this analysis at each of a number of instances in time, correlations between different quantities of web data relating to a respective health-related condition and the demand for resources as evidenced by the historical data regarding the demand for resources of the health care organization that corresponds in time to the historical web data at each of the different instances may be determined. As such, the processing circuitry of an example embodiment is configured to determine a relationship between a change in the quantity of the web data relating to the respective health-related condition based upon historical web data and a change in the demand for resources of the health care organization based upon the historical data regarding the demand for resources of the health care organization.

The relationship may be determined, for example, based upon a regression analysis of the correlations between different quantities of web data and corresponding levels of demand for resources of the health care organization that are determined at various instances in time based upon historical data. For example, the processing circuitry 12 may be configured to determine, such as based upon a regression analysis of prior correlations between the quantity of web data relating to a respective health-related condition and the demand for resources of the health care organization, that a 50% increase in the quantity of web data relating to a respective health-related condition may correspondingly cause an increase in the demand for resources of the health care organization due to treatment of the patients for the respective health-related condition, albeit a smaller percentage change than that reflected by the change in the quantity of web data, such as a 10% increase in the demand for resources of the health care organization due to treatment of the patients having the respective health-related condition. In this regard, the demand for resources of the health care organization that is due to the treatment of patients having the health-related condition includes both the resources utilized to treat the patients having the health-related condition and the resources utilized to replenish the supplies of the health care organization that were depleted by the treatment of the patients having the health-related condition. By way of another example, the processing circuitry may be configured to determine, such as based upon a regression analysis of prior correlations between the quantity of web data relating to a respective health-related condition and the demand for resources of the health care organization, that a 30% decrease in the quantity of web data relating to a respective health-related condition may correspondingly cause a decrease in the demand for resources of the health care organization due to the lack of need for treatment of patients for the respective health-related condition, albeit a smaller percentage change than that reflected by the change in the quantity of web data, such as a 5% decrease in the demand for resources of the health care organization due to lack of need for treatment of patients for the respective health-related condition. Although the change in demand for resources of the health care organization due to treatment of patients having a respective health-related condition was a smaller percentage than the change in the quantity of web data related to the respective health-related condition in the foregoing examples, the percentage change may be larger (or equal) in other embodiments such that the change in demand for resources of the health care organization due to treatment of patients having a respective health-related condition is a larger (or equal) percentage than the change in the quantity of web data related to the respective health-related condition.

The processing circuitry 12 of an example embodiment is configured to determine the correlation, not simply based upon a change in the quantity of demand, but also by determining the correlation based upon a latency between the timing associated with the web data and the timing associated with the demand for resources of the health care organization. In this regard, the timing associated with the web data is the time at which the web data was authored, posted or otherwise originated, while the timing associated with the demand for resources of the health care organization is the time at which the demand for resources occurred. The timing associated with the web data generally precedes the timing associated with the demand for resources of the health care organization. By way of example, a potential patient may post or tweet various social media messages relating to having symptoms of measles a day or more in advance of being admitted to a hospital to treat the measles.

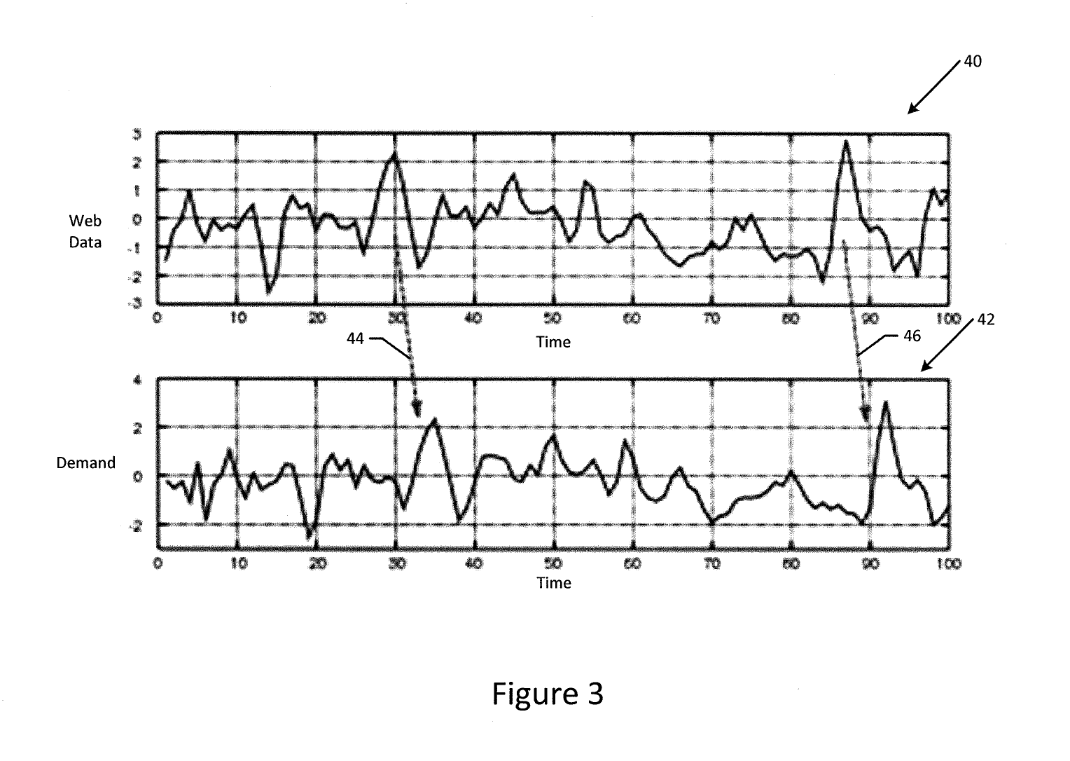

In order to determine the correlation based upon the latency, the processing circuitry 12 of an example embodiment is configured to determine a delay between a change in the web data relating to a respective health-related condition and a change in the demand for resources of the health care organization related to the same health-related condition. In one example, this determination of the delay is based upon a correlation drawn between historical web data and historical data regarding the demand for resources of the health care organization. For example, the processing circuitry may be configured to determine the time lapse between a marked increase in the web data relating to a respective health-related condition and a corresponding increase, albeit perhaps of a lesser degree, for resources of the health care organization due to treatment of the respective health-related condition. By way of example, FIG. 3 provides a graphical representation 40 of the web data relating to a respective health care condition over a period of 100 days. The demand for resources of the health care organization, such as resources of the health care organization to address the respective health care condition, is also depicted by the graphical representation 42. As shown by comparison of the graph representations, the demand for resources of the health care organization related to the respective health-related condition generally follows the same pattern as the web data, albeit with a temporal delay.

As shown, for example, by correlation 44, the marked increase in the web data related to a respective health-related condition on day 30 corresponds to an increase in demand for resources of the health care organization on day 35, thereby evidencing a delay of 5 days. As similarly shown by correlation 46, the marked increase in the web data relating to a respective health-related condition on day 87 corresponds to a similar marked increase in the demand for resources of the health care organization relating to the respective health-related condition on day 92, another delay of about 5 days. Thus, based upon a comparison between the historical web data and the historical data evidencing demand for resources of the health care organization due to a respective health-related condition, the processing circuitry 12 may determine the latency therebetween, such as a delay of 5 days in the example of FIG. 3. Although the processing circuitry may determine the latency in various manners including by establishing a correlation between the historical web data and the historical data evidencing demand for resources of the health care organization due to a respective health-related condition, the processing circuitry of an example embodiment may employ machine learning and/or artificial intelligence in order to determine the correlation between the web data belonging to a respective health-related condition and the demand for resources of the health care organization due to the same health-related condition including, for example the latency therebetween.

Based upon the correlation, including the latency, between a change in the quantity of the web data relating to the respective health-related condition based upon historical web data and a change in the demand for resources of the health care organization based upon the historical data regarding the demand for resources of the health care organization, the processing circuitry 12 is configured to generate a predictive model for resource planning at the health care organization. As described below, the predictive model may be used to predict a required modification of resources at some point in the future (as defined by the application of the latency going forward) based on the web data for a particular condition. Thus, the correlation that is determined from an analysis of historical web data and historical data regarding the demand for resources of the health care organization is applied by the processing circuitry to allow for resource planning at a point in the future based upon current or recent web data. The time lapse between the current or recent web data and the time in the future for which the resource planning is relevant may be based upon the latency, as determined from the historical web data and historical data regarding the demand for resources of the health care organization.

The health care enterprise resource planning system 10 of an example embodiment also includes means, such as the processing circuitry 12, the processor 14 or the like, for modifying the resources of the health care organization at least partially based upon the correlation, including the latency, between the web data relating to the one or more health-related conditions and the demand for resources of the health care organization, such as the demand for resources of the health care organization due to treatment of the respective health-related condition. See block 36 of FIG. 2. In regards to the modification of the resources of the health care organization, the processing circuitry 12 of an example embodiment is configured to modify the contents of memory 16 by storing the modified amounts of the resources of the health care organization that are to be allotted.

In instances in which the processing circuitry 12 has determined that the demand for resources of the health care organization to treat a respective health-related condition will increase with the timing of the increase being based upon the latency between the change in the quantity of web data relating to the respective health-related condition and the change in the demand for resources of the health care organization, the processing circuitry may be configured to allot an increased amount of resources of the health care organization at a future time that is defined based upon the latency. Conversely, in instances in which the processing circuitry has determined that the demand for resources of the health care organization to treat a respective health-related condition will decrease with the timing of the decrease being based upon the latency between the change in the quantity of web data relating to the respective health-related condition and the change in the demand for resources of the health care organization, the processing circuitry may be configured to allot a decreased amount of resources of the health care organization at a future time that is defined based upon the latency.

As such, the allotment of resources of the health care organization to treat the respective health-related condition may be tailored based upon the more temporally relevant web data so as to better match the actual demand. In this way, a present change in web data relating to a health condition can be detected, and the change may be applied by the predictive model to determine a required change in resources at a point in the future based on the determined latency for the particular health condition. By more accurately allotting the resources of the health care organization, the functioning of the health care enterprise resource planning system 10 and method is improved from a technical standpoint as the health care organization will make fewer modifications in the allotment of resources at the time of or following the inception of the demand, thereby improving the performance of the health care enterprise resource planning system and method since modifications at the time of or following the inception of the demand must be performed in an urgent manner which results in increased demand in terms of processing and communications resources than those required in an instance in which the allotment of resources is tailored in advance based on the web data.

As described above, FIG. 3 is a flowchart of a health care enterprise resource planning system, method and computer program product according to example embodiments of the invention. It will be understood that each block of the flowchart, and combinations of blocks in the flowchart, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which embody the procedures described herein may be stored by one or more memory devices 16 and executed by processor 14 of the computer system of FIG. 1. In some embodiments, the computer program instructions comprising the computer program product(s) which embody the procedures described above may be stored by memory devices of a plurality of computing devices. As will be appreciated, any such computer program product may be loaded onto a computer or other programmable apparatus to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product comprises an article of manufacture which implements the function specified in the flowchart block(s). The computer program instructions of one or more computer program products may also be loaded onto a computer or other programmable apparatus to cause a series of operations to be performed on the computer or other programmable apparatus to produce a computer-implemented process such that the instructions which execute on the computer or other programmable apparatus implement the functions specified in the flowchart block(s).

Accordingly, blocks or steps of the flowchart support combinations of means for performing the specified functions and combinations of steps for performing the specified functions. It will also be understood that one or more blocks of the flowchart, and combinations of blocks in the flowchart, may be implemented by special purpose hardware-based computer systems which perform the specified functions or steps, or combinations of special purpose hardware and computer program product(s).

The above described functions may be carried out in many ways. For example, any suitable means for carrying out each of the functions described above may be employed to carry out embodiments of the invention. In one embodiment, a suitably configured processing circuitry 12 may provide all or a portion of the elements of the invention. In another embodiment, all or a portion of the elements of the invention may be configured by and operate under control of a computer program product. The computer program product for performing the methods of embodiments of the invention includes a computer-readable storage medium, such as the non-volatile storage medium, and computer-readable program code portions, such as a series of computer instructions, embodied in the computer-readable storage medium.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

That which is claimed:

1. A health care enterprise resource planning system comprising processing circuitry configured to:
receive web data relating to one or more health-related conditions that are capable of modifying a demand for resources of a health care organization;
determine a correlation between the web data relating to the one or more health-related conditions and the demand for resources of the health care organization; and
modify an allotment of the resources of the health care organization at least partially based upon the correlation between the web data relating to the one or more health-related conditions and the demand for resources of the health care organization.

2. A health care enterprise resource planning system according to claim 1 wherein the processing circuitry is configured to receive web data by filtering the web data based upon a geographical location associated with the web data relative to a service area of the health care organization such that the web data upon which the correlation is determined is web data that is associated with a geographical location within the service area of the health care organization.

3. A health care enterprise resource planning system according to claim 1 wherein the processing circuitry is configured to receive web data by receiving search results based upon one or more search queries relating to the one or more health-related conditions.

4. A health care resource planning system according to claim 1 wherein the processing circuitry is configured to receive web data by identifying information provided via social media relating to the one or more health-related conditions.

5. A health care resource planning system according to claim 1 wherein the processing circuitry is configured to determine the correlation based upon a latency between timing associated with the web data and timing associated with the demand for resources of the health care organization, and wherein the processing circuitry is configured to modify the allotment of the resources of the health care organization at least partially based upon the latency between the web data relating to the one or more health-related conditions and the demand for resources of the health care organization.

6. A health care enterprise resource planning system according to claim 5 wherein the processing circuitry is configured to determine the correlation based upon the latency by determining a delay between a change in the web data relating to the one or more health-related conditions and a change in the demand for resources of the health care organization based upon historical web data and historical data regarding the demand for resources of the health care organization.

7. A health care enterprise resource planning system according to claim 1 wherein the processing circuitry is configured to determine the correlation by determining a relationship between a change in a quantity of the web data relating to the one or more health-related conditions and a change in the demand for resources of the health care organization based upon historical web data and historical data regarding the demand for resources of the health care organization.

8. A health care enterprise resource planning system according to claim 1 wherein the processing circuitry is configured to modify the allotment of the resources of the health care organization by modifying the allotment of at least one of human resources or physical resources of the health care organization that are to be utilized to treat the one or more health-related conditions.

9. A method of performing enterprise resource planning for a health care organization based at least partially upon web data, the method comprising:
receiving web data relating to one or more health-related conditions that are capable of modifying a demand for resources of a health care organization;
determining a correlation between the web data relating to the one or more health-related conditions and the demand for resources of the health care organization; and
modifying an allotment of the resources of the health care organization at least partially based upon the correlation between the web data relating to the one or more health-related conditions and the demand for resources of the health care organization.

10. A method according to claim 9 wherein receiving web data comprises filtering the web data based upon a geographical location associated with the web data relative to a service area of the health care organization such that the web data upon which the correlation is determined is web data that is associated with a geographical location within the service area of the health care organization.

11. A method according to claim 9 wherein receiving web data comprises receiving search results based upon one or more search queries relating to the one or more health-related conditions.

12. A method according to claim 9 wherein receiving web data comprises identifying information provided via social media relating to the one or more health-related conditions.

13. A method according to claim 9 wherein determining the correlation comprises determining the correlation based upon a latency between timing associated with the web data and timing associated with the demand for resources of the health care organization, and wherein modifying the allotment of the resources of the health care organization comprises modifying the allotment of the resources of the health care organization at least partially based upon the latency between the web data relating to the one or more health-related conditions and the demand for resources of the health care organization.

14. A method according to claim 13 wherein determining the correlation based upon the latency comprises determining a delay between a change in the web data relating to the one or more health-related conditions and a change in the demand for resources of the health care organization based upon historical web data and historical data regarding the demand for resources of the health care organization.

15. A method according to claim 9 wherein determining the correlation comprises determining a relationship between a change in a quantity of the web data relating to the one or more health-related conditions and a change in the demand for resources of the health care organization based upon historical web data and historical data regarding the demand for resources of the health care organization.

16. A method according to claim 9 wherein modifying the allotment of the resources of the health care organization comprises modifying the allotment of at least one of human resources or physical resources of the health care organization that are to be utilized to treat the one or more health-related conditions.

17. A method comprising:
receiving web data relating to one or more health-related conditions that are capable of modifying a demand for resources of a health care organization;
determining a change in quantity of the web data related to a respective health-related condition;
determining a corresponding change in demand for resources of the health care organization that are required due to treatment of patients having the respective health-related condition based upon a predictive model and the change in the quantity of the web data relating to the respective health-related condition; and
modifying the allotment of the resources of the health care organization based on the corresponding change.

18. A method according to claim 17 further comprising using the predictive model to determine a latency between the change in the quantity of the web data and the corresponding change in the demand for resources of the health care organization, and wherein modifying the allotment of resources of the health care organization further comprises modifying the resources at a future time based on determined latency.

19. A method according to claim 18 wherein using the predictive model to determine the latency comprises using the predictive model to determine a delay between a change in the web data relating to the one or more health-related conditions and a change in the demand for resources of the health care organization based upon historical web data and historical data regarding the demand for resources of the health care organization.

20. A method according to claim 17 wherein receiving web data comprises filtering the web data based upon a geographical location associated with the web data relative to a service area of the health care organization such that the web data that changes in quantity is web data that is associated with a geographical location with the service area of the health care organization.
